SEQUENCE LISTING (28 sequences)

SEQ ID NO: 1 - DNA, 19 bp, Artificial Sequence
  Synthetic Sequence
gatgcacatccgaagccac

SEQ ID NO: 2 - RNA, 19 nt, Artificial Sequence
  Synthetic Seqeunce
gaugcacauccgaagccac

SEQ ID NO: 3 - RNA, 17 nt, Artificial Sequence
  Synthetic Sequence
gaugcacauccgaagcc

SEQ ID NO: 4 - RNA, 18 nt, Artificial Sequence
  Synthetic Sequence
gaugcacauccgaagcca

SEQ ID NO: 5 - RNA, 20 nt, Artificial Sequence
  Synthetic Sequence
gaugcacauccgaagccaca

SEQ ID NO: 6 - RNA, 21 nt, Artificial Sequence
  Synthetic Sequence
gaugcacauccgaagccacac

SEQ ID NO: 7 - DNA, 29 bp, Artificial Sequence
  Synthetic Sequence
ucaagaugcacauccgaagccacacgcnn

SEQ ID NO: 8 - DNA, 27 bp, Artificial Sequence
  Synthetic Sequence
ucaagaugcacauccgaagccacacgc

SEQ ID NO: 9 - DNA, 64 bp, Artificial Sequence
  Synthetic Sequence
gatccccgatgcacatccgaagccacttcaagagagtggcttcggatgtgcatctttttggaaa

SEQ ID NO: 10 - DNA, 19 bp, Artificial Sequence
  Synthetic Sequence
gtggcttcggatgtgcatc

SEQ ID NO: 11 - DNA, 9 bp, Artificial Sequence
  Synthetic Sequence
ttcaagaga

SEQ ID NO: 12 - DNA, 64 bp, Artificial Sequence
  Synthetic Sequence
agcttttccaaaaagatgcacatccgaagccactctcttgaagtggcttcggatgtgcatcggg

SEQ ID NO: 13 - DNA, 9 bp, Artificial Sequence
  Synthetic Sequence
tctcttgaa

SEQ ID NO: 14 - RNA, 49 nt, Artificial Sequence
  Synthetic Sequence
gaugcacauccgaagccacuucaagagaguggcuucggaugugcaucuu

SEQ ID NO: 15 - RNA, 21 nt, Artificial Sequence
  Synthetic Sequence
guggcuucggaugugcaucuu

SEQ ID NO: 16 - DNA, 19 bp, Artificial Sequence
  Synthetic Sequence
caaggaatacctcagcctg

SEQ ID NO: 17 - DNA, 19 bp, Artificial Sequence
  Synthetic Sequence
gcgagctgcaggactctaa

SEQ ID NO: 18 - RNA, 22 nt, Artificial Sequence
  Synthetic Sequence
gaugcacauccgaagccacacg

SEQ ID NO: 19 - RNA, 23 nt, Artificial Sequence
  Synthetic Sequence
gaugcacauccgaagccacacgc

SEQ ID NO: 20 - DNA, 19 bp, Artificial Sequence
  Synthetic Sequence
gatgcacattcgaacccac

SEQ ID NO: 21 - DNA, 19 bp, Artificial Sequence
  Synthetic Sequence
gatgcatattcggacccac

SEQ ID NO: 22 - RNA, 17 nt, Artificial Sequence
  Synthetic Sequence
ugcacauccgaagccac

SEQ ID NO: 23 - RNA, 18 nt, Artificial Sequence
  Synthetic Sequence
augcacauccgaagccac

SEQ ID NO: 24 - RNA, 20 nt, Artificial Sequence
  Synthetic Sequence
agaugcacauccgaagccac

SEQ ID NO: 25 - RNA, 21 nt, Artificial Sequence
  Synthetic Sequence
aagaugcacauccgaagccac

SEQ ID NO: 26 - RNA, 22 nt, Artificial Sequence
  Synthetic Sequence
caagaugcacauccgaagccac

SEQ ID NO: 27 - RNA, 23 nt, Artificial Sequence
  Synthetic Sequence
ucaagaugcacauccgaagccac

SEQ ID NO: 28 - DNA, 1708 bp, Homo Sapiens
ggcacggcctagcgagtggttcttctgcgctactgctgcgcgaatcggcgaccccagtgcctcgaccactatgccgcgctctttcctcgtcaggaagccctccgaccccaatcggaagcctaactacagcgagctgcaggactctaatccagagtttaccttccagcagccctacgaccaggcccacctgctggcagccatcccacctccggagatcctcaaccccaccgcctcgctgccaatgctcatctgggactctgtcctggcgccccaagcccagccaattgcctgggcctcccttcggctccaggagagtcccagggtggcagagctgacctccctgtcagatgaggacagtgggaaaggctcccagccccccagcccaccctcaccggctccttcgtccttctcctctacttcagtctcttccttggaggccgaggcctatgctgccttcccaggcttgggccaagtgcccaagcagctggcccagctctctgaggccaaggatctccaggctcgaaaggccttcaactgcaaatactgcaacaaggaatacctcagcctgggtgccctcaagatgcacatccgaagccacacgctgccctgcgtctgcggaacctgcgggaaggccttctctaggccctggctgctacaaggccatgtccggacccacactggcgagaagcccttctcctgtccccactgcagccgtgccttcgctgaccgctccaacctgcgggcccacctccagacccactcagatgtcaagaagtaccagtgccaggcgtgtgctcggaccttctcccgaatgtccctgctccacaagcaccaagagtccggctgctcaggatgtccccgctgaccctcgaggctccctcttcctctccatacctgcccctgcctgacagccttccccagctccagcaggaaggaccccacatccttctcactgccatggaattccctcctgagtgccccacttctggccacatcagccccacaggactttgatgaagaccattttctggttctgtgtcctctgcctgggctctggaagaggccttcccatggccatttctgtggagggagggcagctggcccccagccctgggggattcctgagctggcctgtctgcgtgggtttttgtatccagagctgtttggatacagctgctttgagctacaggacaaaggctgacagactcactgggaagctcccaccccactcaggggaccccactcccctcacacacacccccccacaaggaaccctcaggccaccctccacgaggtgtgactaactatgcaataatccacccccaggtgcagccccagggcctgcggaggcggtggcagactagagtctgagatgccccgagcccaggcagctatttcagcctcctgtttggtggggtggcacctgtttcccgggcaatttaacaatgtctgaaaagggactgtgagtaatggctgtcacttgtcgggggcccaagtggggtgctctggtctgaccgatgtgtctcccagaactattctgggggcccgacaggtgggcctgggaggaagatgtttacatttttaaaggtacactggtatttatatttcaaacattttgtatcaaggaaacgttttgtatagttatatgtacagtttattgatattcaataaagcagttaatttatatattaaaaaaaaaaaaaaaaaa

(19) United States
(12) Patent Application Publication
(10) Pub. No.: US 2006/0003956 A1
(43) Pub. Date: Jan. 5, 2006

(54) Materials and methods for the derepression of the E-cadherin promoter

(76) Inventors: David Olmeda Casadome, Madrid (ES); Hector Peinado Selgas, Madrid (ES); Francisco Portillo Perez, Madrid (ES); Maria Angela Nieto Toledano, Alicante (ES); Amparo Cano Garcia, Madrid (ES)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/071,025

(22) Filed: Mar. 3, 2005

Related U.S. Application Data
(60) Provisional application No. 60/549,740, filed on Mar. 3, 2004.

(51) Int. Cl. A61K 48/00; C07H 21/02

(52) U.S. Cl. 514/44; 536/23.1

(57) ABSTRACT

Nucleic acid is provided that is capable, when suitably introduced into or expressed within a mammalian cell that otherwise expresses the transcription factor Snail, of suppressing Snail expression by RNAi. Preferred target sequences of Snail mRNA are gatgcacatccgaagccac (SEQ ID NO:1) and sequences overlapping therewith. Snail expression is associated with repression of the E-cadherin promoter and tumour progression.

### FIELD OF THE INVENTION

The present invention relates to materials and methods for the suppression of expression of Snail protein, and particularly although not exclusively to nucleic acids for use in derepression of the E-cadherin promoter.

### BACKGROUND TO THE INVENTION

Snail

Maintenance of stable cell-cell contacts and cell polarity is an essential requirement for the functionality and homeostasis of epithelial tissues in the adult organism. This strict tissue organisation is lost during the progression of epithelial tumours (carcinomas) and is particularly evident at the invasion stage when tumour cells dissociate from the primary tumour and acquire the ability to transverse the basement membrane that delimits the epithelial tissues from the adjacent connective tissues (Behrens et al., 1992; Stetler-Stevenson et al., 1993).

The E-cadherin/catenin complexes represent the main adhesion system responsible for the maintenance of cell-cell contacts in epithelial tissues (Takeichi, 1995; Huber et al., 1996). Downregulation of E-cadherin expression or functional perturbations of the E-cadherin/catenin complexes have been found to occur very frequently during the progression of carcinomas (Takeichi, 1993; Birchmeier and Behrens, 1994; Christofori and Semb, 1999). Indeed, loss of E-cadherin expression has been shown to be responsible for the loss of intercellular adhesion occurring during invasion (Perl et al., 1998). As a consequence, during the invasive process, tumour cells not only lose their cell-cell adhesion properties but also frequently undergo profound changes in their phenotype known as epithelial-mesenchymal transitions (EMTs) (Behrens et al., 1992; Christofori and Semb, 1999), a process reminiscent of EMT occurring in development (Thiery, 2002; Nieto, 2002).

The molecular bases of the E-cadherin downregulation during tumour progression have started to be elucidated in recent years. The present evidence indicates that silencing of E-cadherin expression may involve genetic and epigenetic changes (Christofori and Semb, 1999). Among them, hypermethylation of the E-cadherin promoter and transcriptional repression have emerged as predominant mechanisms in most carcinomas (Risinger et al., 1994; Yoshiura et al., 1995; Henning et al., 1996; Giroldi et al., 1997; Hajra et al., 1999; Rodrigo et al., 1999; Tamura et al., 2000; Cheng et al., 2001).

Previous studies on the mouse E-cadherin promoter identified a palindromic element, containing two adjacent E-boxes, as the major repressor element (Hennig et al, 1996; Faraldo et al., 1997; Rodrigo et al., 1999), and similar E-boxes are found in the human E-cadherin promoter (Henning et al., 1996; Comijn et al., 2001). Several independent factors, Snail, Slug, E47bHLH, ZEB-1 (8EF-1), and SIP-1 (ZEB-2) have been recently characterized as transcriptional repressors of E-cadherin acting through interaction with the E-pal element, or equivalent E-boxes, of the proximal promoter (Cano et al., 2000; Batlle et al., 2000; Perez-Moreno et al., 2001; Grooteclaes and Frisch, 2000; Comijn et al., 2001; Hajra et al., 2002; Bolos et al. 2003).

The zinc finger factors Snail and Slug belong to the Snail superfamily of transcriptional repressors with a major role in triggering EMT during development of diverse species from Drosophila to mammals (reviewed in Nieto, 2002). Indeed, generation of Snail knock-out mice has provided compelling evidence for its major implication in EMT as an E-cadherin repressor, since Snail null mice are unable to undergo a complete gastrulation process, generating an aberrant mesodermal layer which maintains E-cadherin expression (Carver et al., 2001).

Moreover, Snail expression has been detected in different human carcinoma and melanoma cell lines (Cano et al., 2000; Batlle et al., 2000; Cheng et al., 2001; Poser et al, 2001; Yokohama et al., 2001). More importantly, Snail is expressed at the invasive front of epidermoid carcinomas (Cano et al., 2000) and in de-differentiated invasive ductal breast carcinomas and hepatocarcinomas (Blanco et al., 2002; Sugimachi et al., 2003).

The participation of E47 in EMT in development is also supported from the expression pattern detected in early mouse embryos (Perez-Moreno et al., 2001). Snail, Slug and E-47 factors induce a full EMT when overexpressed in epithelial MDCK cells and led to the acquisition of an invasive phenotype (Cano et al., 2000; Perez-Moreno et al. 2001; Bolos et al., 2003).

However, binding studies of the three factors to the specific E-pal element of the E-cadherin promoter have established that Snail binds with a much higher affinity than E47bHLH and Slug to the repressor element (Bolos et al. 2003, co-authored by the inventors).

RNAi

RNA interference (RNAi) is a naturally occurring process in which double-stranded RNA (dsRNA) duplexes cause the suppression of gene translation by targeting mRNA.

In mammalian cells, the introduction of long dsRNA leads to non-specific repression of translation of many proteins, via DNA-dependent protein kinase (PKR). Recently, however, it was discovered that short dsRNA duplexes (known as small interfering RNA, or siRNA) barely activate PKR and allow specific suppression of translation of a target mRNA via RNAi.

Typically, the siRNA technique uses a synthetic double-stranded nucleic acid duplex, in which each strand is from 19-23 nucleotides in length, with 2 nucleotides in each strand forming a 3′ overhang. These 3′ overhangs may be RNA or DNA whereas the double-stranded region is entirely RNA. Typically, the 3′ overhangs are symmetrical and have the sequence 5′-UU-3′ or 5′-UG-3′ (5′-TT-3′ or 5′-TG-3′ if the 3′ overhang is DNA).

It is also possible to obtain the same effect by directing transcription of an RNA molecule that self-hybridises, wherein the RNA molecule has two complementary portions that are joined via a sequence that forms a hairpin structure. The enzyme DICER then cleaves the hairpin structure, leaving a double-stranded RNA duplex like those described above, again with the 3′ overhangs. Such self-hybridising RNA is also known as shRNA (short hairpin RNA) that, after DICER processing, will generate a double-stranded RNAi (siRNA).

The advantages of RNAi over other gene-targeting strategies such as anti-sense oligonucleotides include its relative specificity, its enhanced efficacy (only nanomolar quantities of siRNA or DNA encoding shRNA are required for efficient gene-silencing), and the fact that siRNA treatment feeds into a natural RNAi pathway that is inherent to all cells.

Nevertheless, the success of gene-silencing by RNAi can be highly variable depending on the gene target, the target sequence, and the cell type being targeted.

siRNA Directed Against Snail

To the inventors' knowledge, siRNA (in the form of duplex RNA) has been used to target Snail in two papers to-date, Fujita et al. (2003) Cell 113:207-219 and Espineda et al. (2004) Mol. Biol. Cell. (in press, published online on 29 Dec. 2003). In both cases, decrease of Snail mRNA levels was tested by RT-PCR. The sequence of the oligonucleotide is identical in both papers, corresponding to positions 129-147 of human Snail mRNA;
5′-GCGAGCTGCAGGACTCTAA-3′ (SEQ ID NO:17).

This differs from the target sequence of the present invention.

### SUMMARY OF THE INVENTION

The inventors have applied the technique of RNAi in vitro in an attempt to suppress the expression of Snail. Despite the intrinsic unpredictability of the efficacy of this approach they surprisingly obtained specific and highly efficient inhibition of the expression of Snail mRNA.

Moreover, despite the presence of Slug protein in the cells tested, stable and transient expression of Snail siRNA also led to dramatic derepression of the E-cadherin promoter.

At its most general, the invention provides nucleic acid that is capable, when suitably introduced into or expressed within a mammalian cell that otherwise expresses Snail, of suppressing Snail expression by RNAi.

The nucleic acid has substantial sequence identity to a portion of human Snail mRNA, as defined in GenBank accession no. NM_{—}005985 (version NM_{—}005985.2; GI:18765740), or the complementary sequence to said mRNA.

The nucleic acid may be a double-stranded siRNA. (As the skilled person will appreciate, and as explained further below, a siRNA molecule may include a short 3′ DNA sequence also.)

Alternatively, the nucleic acid may be a DNA (usually double-stranded DNA) which, when transcribed in a mammalian cell, yields an RNA having two complementary portions joined via a spacer, such that the RNA takes the form of a hairpin when the complementary portions hybridise with each other. In a mammalian cell, the hairpin structure may be cleaved from the molecule by the enzyme DICER, to yield two distinct, but hybridised, RNA molecules.

Preferably the nucleic acid is generally targeted to the sequence:
gatgcacatccgaagccac (SEQ ID NO:1).

This sequence appears in the mRNA of human Snail at positions 580-598 of GenBank accession no. NM_{—}005985 (version NM_{—}005985.2; GI:18765740), reproduced below (SEQ ID NO: 28).

| | |
|---|---|
| 1 | ggcacggcct agcgagtggt tcttctgcgc tactgctgcg cgaatcggcg accccagtgc |
| 61 | ctcgaccact atgccgcgct ctttcctcgt caggaagccc tccgacccca atcggaagcc |
| 121 | taactacagc gagctgcagg actctaatcc agagtttacc ttccagcagc cctacgacca |
| 181 | ggcccacctg ctggcagcca tcccacctcc ggagatcctc aaccccaccg cctcgctgcc |
| 241 | aatgctcatc tgggactctg tcctggcgcc ccaagcccag ccaattgcct gggcctccct |
| 301 | tcggctccag gagagtccca gggtggcaga gctgacctcc ctgtcagatg aggacagtgg |
| 361 | gaaaggctcc cagcccccca gcccaccctc accggctcct tcgtccttct cctctacttc |
| 421 | agtctcttcc ttggaggccg aggcctatgc tgccttccca ggcttgggcc aagtgcccaa |
| 481 | gcagctggcc cagctctctg aggccaagga tctccaggct cgaaaggcct tcaactgcaa |
| 541 | atactgcaac aaggaatacc tcagcctggg tgccctcaag atgcacatcc gaagccacac |
| 601 | gctgccctgc gtctgcggaa cctgcgggaa ggccttctct aggccctggc tgctacaagg |
| 661 | ccatgtccgg acccacactg gcgagaagcc cttctcctgt ccccactgca gccgtgcctt |
| 721 | cgctgaccgc tccaacctgc gggcccacct ccagacccac tcagatgtca agaagtacca |
| 781 | gtgccaggcg tgtgctcgga ccttctcccg aatgtccctg ctccacaagc accaagagtc |
| 841 | cggctgctca ggatgtcccc gctgaccctc gaggctccct cttcctctcc atacctgccc |
| 901 | ctgcctgaca gccttcccca gctccagcag gaaggacccc acatccttct cactgccatg |
| 961 | gaattccctc ctgagtgccc cacttctggc cacatcagcc ccacaggact ttgatgaaga |
| 1021 | ccattttctg gttctgtgtc ctctgcctgg gctctggaag aggccttccc atggccattt |
| 1081 | ctgtggaggg agggcagctg gcccccagcc ctgggggatt cctgagctgg cctgtctgcg |
| 1141 | tgggtttttg tatccagagc tgtttggata cagctgcttt gagctacagg acaaaggctg |
| 1201 | acagactcac tgggaagctc ccaccccact caggggaccc cactcccctc acacacaccc |
| 1261 | ccccacaagg aaccctcagg ccaccctcca cgaggtgtga ctaactatgc aataatccac |
| 1321 | ccccaggtgc agccccaggg cctgcggagg cggtggcaga ctagagtctg agatgccccg |
| 1381 | agcccaggca gctatttcag cctcctgttt ggtggggtgg cacctgtttc ccgggcaatt |
| 1441 | taacaatgtc tgaaaaggga ctgtgagtaa tggctgtcac ttgtcggggg cccaagtggg |
| 1501 | gtgctctggt ctgaccgatg tgtctcccag aactattctg qgggcccgac aggtgggcct |
| 1561 | gggaggaaga tgtttacatt tttaaaggta cactggtatt tatatttcaa acattttgta |
| 1621 | tcaaggaaac gttttgtata gttatatgta cagtttattg atattcaata aagcagttaa |
| 1681 | tttatatatt aaaaaaaaaa aaaaaaaa |

It is identical to the corresponding portion in the mRNA of mouse Snail, at positions 573-591 of GenBank accession no. NM_{—}011427 (version NM_{—}011427.2; GI:31981483).

It varies only slightly (at two positions) from the corresponding portion in the mRNA of mouse Slug, at positions 571-589 of GenBank accession no. NM_{—}011415 (version NM_{—}011415.1; GI:6755575). This has the sequence gatgcacattcgaacccac (SEQ ID NO:20), in which the underlined residues differ from the Snail sequence.

It similarly varies only slightly (at four positions) from the corresponding portion in the mRNA sequence of human Slug, at positions 687-707 of GenBank accession no. NM-003068 (version NM_{—}003068.3; GI:24497625). This has the sequence gatgcatattcggacccac (SEQ ID NO:21), in which the underlined residues differ from the Snail sequence.

Despite the similarity between the Snail and Slug sequences in this region, the work reported in the Examples shows remarkable specificity for the suppression of expression of Snail when this region is targeted using RNAi.

Only single-stranded (i.e. non self-hybridised) regions of an mRNA transcript are expected to be suitable targets for RNAi. It is therefore proposed that other sequences very close in the Snail mRNA transcript to the sequence represented by SEQ ID NO:1 may also be suitable targets for RNAi. Such target sequences are preferably 17-23 nucleotides in length and preferably overlap SEQ ID NO:1 by at least 1, 2, 3, 4, 5, 6, 7, 8, 9, 10, 11, 12, 13, 14, 15, 16, 17, 18 or all 19 nucleotides (at either end of SEQ ID NO:1). More preferably, they include SEQ ID NO:1.

Accordingly, the invention provides nucleic acid that is capable, when suitably introduced into or expressed within a mammalian cell that otherwise expresses Snail, of suppressing Snail expression by RNAi, wherein the nucleic acid is generally targeted to the sequence gatgcacatccgaagccac (SEQ ID NO:1).

By “generally targeted” it is intended that the nucleic acid targets a sequence that overlaps with SEQ ID NO: 1. In particular, the nucleic acid may target a sequence in the mRNA of human Snail that is slightly longer or shorter than SEQ ID NO:1 (preferably from 17-23 nucleotides in length), but is otherwise identical to SEQ ID NO:1.

It is expected that perfect identity/complementarity between the nucleic acid of the invention and the target sequence, although preferred, is not essential. Accordingly, the nucleic acid of the invention may include a single mismatch compared to the mRNA of human Snail. It is expected, however, that the presence of even a single mismatch is likely to lead to reduced efficiency, so the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the invention that include non-natural nucleotides.

### DETAILED DESCRIPTION OF THE PREFERRED EMBODIMENTS

In one embodiment, the nucleic acid (herein referred to as double-stranded siRNA) includes the following double-stranded RNA sequence:

| | |
|---|---|
| 5′ -GAUGCACAUCCGAAGCCAC-3′ | (SEQ ID NO:2) |
| 3′ -CUACGUGUAGGCUUCGGUG-5′ | |

However, it is also expected that slightly shorter or longer sequences directed to the same region of Snail mRNA will also be effective. In particular, it is expected that double-stranded sequences between 17 and 23 bp in length will also be effective.

Accordingly, the nucleic acid of the invention may include a double-stranded RNA sequence selected from the following group:

| | |
|---|---|
| 5′ -GAUGCACAUCCGAAGCC-3′ | (SEQ ID NO:3) |
| 3′ -CUACGUGUAGGCUUCGG-5′ | |
| 5′ -GAUGCACAUCCGAAGCCA-3′ | (SEQ ID NO:4) |
| 3′ -CUACGUGUAGGCUUCGGU-5′ | |
| 5′ -GAUGCACAUCCGAAGCCACA-3′ | (SEQ ID NO:5) |
| 3′ -CUACGUGUAGGCUUCGGUGU-5′ | |
| 5′ -GAUGCACAUCCGAAGCCACAC-3′ | (SEQ ID NO:6) |
| 3′ -CUACGUGUAGGCUUCGGUGUG-5′ | |
| 5′ -GAUGCACAUCCGAAGCCACACG-3′ | (SEQ ID NO:18) |
| 3′ -CUACGUGUAGGCUUCGGUGUGC-5′ | |
| 5′ -GAUGCACAUCCGAAGCCACACGC-3′ | (SEQ ID NO:19) |
| 3′ -CUACGUGUAGGCUUCGGUGUGCG-5′ | |
| 5′ -UGCACAUCCGAAGCCAC-3′ | (SEQ ID NO:22) |
| 3′ -ACGUGUAGGCUUCGGUG-5′ | |
| 5′ -AUGCACAUCCGAAGCCAC-3′ | (SEQ ID NO:23) |
| 3′ -UACGUGUAGGCUUCGGUG-5′ | |
| 5′ -AGAUGCACAUCCGAAGCCAC-3′ | (SEQ ID NO:24) |
| 3′ -UCUACGUGUAGGCUUCGGUG-5′ | |
| 5′ -AAGAUGCACAUCCGAAGCCAC-3′ | (SEQ ID NO:25) |
| 3′ -UUCUACGUGUAGGCUUCGGUG-5′ | |
| 5′ -CAAGAUGCACAUCCGAAGCCAC-3′ | (SEQ ID NO:26) |
| 3′ -GUUCUACGUGUAGGCUUCGGUG-5′ | |
| 5′ -UCAAGAUGCACAUCCGAAGCCAC-3′ | (SEQ ID NO:27) |
| 3′ -AGUUCUACGUGUAGGCUUCGGUG-5′ | |

As previously mentioned, the nucleic acid of the invention may have a single mismatch compared to the mRNA of human Snail. Accordingly, the invention also includes sequences having a single base pair substitution compared to any one of SEQ ID NOs 2, 3-6, 18, 19 and 22-27.

The strands that form the double-stranded RNA may have short 3′ dinucleotide overhangs, which may be DNA or RNA. The use of a 3′ DNA overhang has no effect on siRNA activity compared to a 3′ RNA overhang, but reduces the cost of chemical synthesis of the nucleic acid strands (Elbashir et al., 2001c). For this reason, DNA dinucleotides may be preferred.

When present, the dinucleotide overhangs may be symmetrical to each other, though this is not essential. Indeed, the 3′ overhang of the sense (upper) strand is irrelevant for RNAi activity, as it does not participate in mRNA recognition and degradation (Elbashir et al., 2001a, 2001b, 2001c)

A -TT (or -UU) antisense (lower strand) overhang is complementary to the AA motif at positions 578-9 of the human Snail mRNA sequence, immediately upstream of SEQ ID NO:1. While RNAi experiments in Drosophila show that antisense 3′ overhangs may participate in mRNA recognition and targeting (Elbashir et al. 2001c), 3′ overhangs do not appear to be necessary for RNAi activity of siRNA in mammalian cells. Incorrect annealing of 3′ overhangs is therefore thought to have little effect in mammalian cells (Elbashir et al. 2001c; Czauderna et al. 2003).

Any dinucleotide overhang may therefore be used in the antisense strand of the siRNA. Nevertheless, the dinucleotide is preferably -UU or -UG (or -TT or -TG if the overhang is DNA), more preferably -UU (or -TT). The -UU (or -TT) dinucleotide overhang is most effective and is consistent with (i.e. capable of forming part of) the RNA polymerase III end of transcription signal. Accordingly, this dinucleotide is most preferred. The dinucleotides AA, CC and GG may also be used, but are less effective and consequently less preferred.

Moreover, the 3′ overhangs may be omitted entirely from the siRNA.

Accordingly, double-stranded siRNAs of the invention are preferably of the following structure:

| | |
|---|---|
| 5′- X-Y -3′ | (SEQ ID NO:7) |
| 3′- Y′-X′ -5′ | |

wherein:
X is the RNA sequence 5′-W-UGCACAUCCGAAGCC-Z-3′ (SEQ ID NO:8), or an RNA sequence differing therefrom by a single substitution;
W is absent or is an RNA sequence selected from the group consisting of A, GA, AGA, AAGA, CAAGA, or UCAAGA, preferably GA;
Z is absent or is an RNA sequence selected from the group consisting of A, AC, ACA, ACAC, ACACG, or ACACGC, preferably AC;
W and Z together have between 2 and 8 nucleotides;
X′ is the reverse complement of X; and
Y and Y′ are absent or are each an RNA or DNA dinucleotide and may be the same or different, preferably the RNA sequence UU or UG or the DNA sequence TT or TG (reading 5′-3′), preferably UU or TT.

The invention also provides single-stranded nucleic acids (herein referred to as single-stranded siRNAs) respectively consisting of a component strand of one of the aforementioned double-stranded nucleic acids, preferably with the 3′-overhangs. The invention also provides kits containing pairs of such single-stranded nucleic acids, which are capable of hybridising with each other in vitro to form the aforementioned double-stranded siRNAs, which may then be introduced into cells.

The nucleic acids of the invention may also incorporate nucleotides other than the ribonucleotides A, C, G and U and the deoxyribonucleotides A, C, G and T. For example, the nucleic acids may incorporate chemically modified nucleotides to improve their stability, efficacy and/or bioavailability. Examples of suitable forms of chemical modification are described in U.S. 2004/0219671A, and documents cited therein. The term “nucleic acid” as used herein encompasses such modifications. Such chemical modification may be applied to any of the preferred sequences disclosed herein.

The invention also provides DNA that, when transcribed in a mammalian cell, yields an RNA (herein also referred to as an shRNA) having two complementary portions which are capable of self-hybridising to produce a double-stranded motif including a sequence selected from the group consisting of SEQ ID NOs: 2-6, 18, 19 and 22-27 or a sequence that differs from any one of the aforementioned sequences by a single base pair substitution.

The complementary portions will generally be joined by a spacer, which has suitable length and sequence to allow the two complementary portions to hybridise with each other. The two complementary (i.e. sense and antisense) portions may be joined 5′-3′ in either order. The spacer will typically be a short sequence, of approximately 4-12 nucleotides, preferably 4-9 nucleotides, more preferably 6-9 nucleotides.

Preferably the 5′ end of the spacer (immediately 3′ of the upstream complementary portion) consists of the nucleotides -UU- or -UG-, again preferably -UU- (though, again, the use of these particular dinucleotides is not essential). A suitable spacer, recommended for use in the pSuper system of OligoEngine (Seattle, Wash., USA) is UUCAAGAGA. In this and other cases, the ends of the spacer may hybridise with each other, elongating the double-stranded motif beyond the exact sequences of SEQ ID NOs 2-6, 18 and 19 by a small number (e.g. 1 or 2) of base pairs.

Similarly, the transcribed RNA preferably includes a 3′ overhang from the downstream complementary portion. Again, this is preferably -UU or -UG, more preferably -UU.

Such shRNA molecules may then be cleaved in the mammalian cell by the enzyme DICER to yield a double-stranded siRNA as described above, in which each strand of the hybridised dsRNA includes a 3′ overhang.

Techniques for the synthesis of the nucleic acids of the invention are of course well known in the art.

The skilled person is well able to construct suitable transcription vectors for the DNA of the invention using well-known techniques and commercially available materials, such as those described in the Examples. In particular, the DNA will be associated with control sequences, including a promoter and a transcription termination sequence.

Of particular suitability are the commercially available pSuper and pSuperior systems of OligoEngine (Seattle, Wash., USA). These use a polymerase-III promoter (H1) and a T₅ transcription terminator sequence that contributes two U residues at the 3′ end of the transcript (which, after DICER processing, provide a 3′ UU overhang of one strand of the siRNA).

Another suitable system is described in Shin et al. (supra), which uses another polymerase-III promoter (U6).

The double-stranded siRNAs of the invention may be introduced into mammalian cells in vitro or in vivo using known techniques, as described below, to suppress expression of Snail.

Similarly, transcription vectors containing the DNAs of the invention may be introduced into tumour cells in vitro or in vivo using known techniques, as described below, for transient or stable expression of RNA, again to suppress expression of Snail.

Generally, suppression of Snail expression in a cell that otherwise would express Snail will also lead to the derepression of the E-cadherin promoter. Preferably the cell is a tumour cell, more preferably a carcinoma or melanoma cell. Preferably the tumour is a breast or colon tumour or melanoma. The carcinoma may for example be a ductal breast carcinoma, a hepatocarcinoma or a colon carcinoma. The term “tumour cell” includes both tumour cells in vivo and in vitro, both as uncultured tissue samples (e.g. tumour biopsies) and cultured tumour cells or cell lines.

The cell may be from any mammal of interest, though human cells, and the treatment of human patients is preferred. Cells from other mammals, such as rodents (e.g. mouse or rat), cat, dog or horse may be of interest for research or veterinary purposes. The term “patients” may therefore encompass non-human mammals.

Accordingly, the invention also provides a method of suppressing Snail expression in a tumour cell, the method comprising administering to the cell a double-stranded siRNA of the invention or a transcription vector of the invention.

Similarly, the invention also provides a method of derepressing the E-cadherin promoter in a tumour cell, the method comprising administering to the cell a double-stranded siRNA of the invention or a transcription vector of the invention.

Similarly, the invention further provides a method of treating tumour, the method comprising administering to a patient having a tumour a double-stranded siRNA of the invention or a transcription vector of the invention.

Such treatment is intended to result in the reduction or limitation of the invasive and/or metastatic properties or potential of the tumour.

The invention further provides the double-stranded siRNAs of the invention and the transcription vectors of the invention, for use in a method of treatment, preferably a method of treating tumour. Preferred tumours are as described above.

The invention further provides the use of the double-stranded siRNAs of the invention and the transcription vectors of the invention in the preparation of a medicament for the treatment of tumour. Again, preferred tumours are as defined above.

The invention further provides a composition comprising a double-stranded siRNA of the invention or a transcription vectors of the invention in admixture with one or more pharmaceutically acceptable carriers. Suitable carriers include lipophilic carriers or vesicles, which may assist in penetration of the cell membrane.

Administration to Cells

Materials and methods suitable for the administration of siRNA duplexes and DNA vectors of the invention are well known in the art and improved methods are under development, given the potential of RNAi technology.

Generally, many techniques are available for introducing nucleic acids into mammalian cells. The choice of technique will depend on whether the nucleic acid is transferred into cultured cells in vitro or in vivo in the cells of a patient. Techniques suitable for the transfer of nucleic acid into mammalian cells in vitro include the use of liposomes, electroporation, microinjection, cell fusion, DEAE dextran and calcium phosphate precipitation. In vivo gene transfer techniques include transfection with viral (typically retroviral) vectors and viral coat protein-liposome mediated transfection (Dzau et al. (2003) Trends in Biotechnology 11, 205-210).

In particular, suitable techniques for cellular administration of the nucleic acids of the invention both in vitro and in vivo are disclosed in the following articles:

General Reviews:

Borkhardt, A. 2002. Blocking oncogenes in malignant cells by RNA interference—new hope for a highly specific cancer treatment? Cancer Cell. 2:167-8
Hannon, G. J. 2002. RNA interference. Nature. 418:244-51.
McManus, M. T., and P. A. Sharp. 2002. Gene silencing in mammals by small interfering RNAs. Nat Rev Genet. 3:737-47.
Scherr, M., M. A. Morgan, and M. Eder. 2003b. Gene silencing mediated by small interfering RNAs in mammalian cells. Curr Med. Chem. 10:245-56.
Shuey, D. J., D. E. McCallus, and T. Giordano. 2002. RNAi: gene-silencing in therapeutic intervention. Drug Discov Today. 7:1040-6.
   Systemic Delivery Using Liposomes:
Lewis, D. L., J. E. Hagstrom, A. G. Loomis, J. A. Wolff, and H. Herweijer. 2002. Efficient delivery of siRNA for inhibition of gene expression in postnatal mice. Nat Genet. 32:107-8.
Paul, C. P., P. D. Good, I. Winer, and D. R. Engelke. 2002. Effective expression of small interfering RNA in human cells. Nat Biotechnol. 20:505-8
Song, E., S. K. Lee, J. Wang, N. Ince, N. Ouyang, J. Min, J. Chen, P. Shankar, and J. Lieberman. 2003. RNA interference targeting Fas protects mice from fulminant hepatitis. Nat Med. 9:347-51.
Sorensen, D. R., M. Leirdal, and M. Sioud. 2003. Gene silencing by systemic delivery of synthetic siRNAs in adult mice. J Mol Biol. 327:761-6.
   Virus Mediated Transfer:
Abbas-Terki, T., W. Blanco-Bose, N. Deglon, W. Pralong, and P. Aebischer. 2002. Lentiviral-mediated RNA interference. Hum Gene Ther. 13:2197-201.
Barton, G. M., and R. Medzhitov. 2002. Retroviral delivery of small interfering RNA into primary cells. Proc Natl Acad Sci U S A. 99:14943-5
Devroe, E., and P. A. Silver. 2002. Retrovirus-delivered siRNA. BMC Biotechnol. 2:15.
Lori, F., P. Guallini, L. Galluzzi, and J. Lisziewicz. 2002. Gene therapy approaches to HIV infection. Am J Pharmacogenomics. 2:245-52.
Matta, H., B. Hozayev, R. Tomar, P. Chugh, and P. M. Chaudhary. 2003. Use of lentiviral vectors for delivery of small interfering RNA. Cancer Biol Ther. 2:206-10
Qin, X. F., D. S. An, I. S. Chen, and D. Baltimore. 2003. Inhibiting HIV-1 infection in human T cells by lentiviral-mediated delivery of small interfering RNA against CCR5. Proc Natl Acad Sci USA. 100:183-8.
Scherr, M., K. Battmer, A. Ganser, and M. Eder. 2003a. Modulation of gene expression by lentiviral-mediated delivery of small interfering RNA. Cell Cycle. 2:251-7
Shen, C., A. K. Buck, X. Liu, M. Winkler, and S. N. Reske. 2003. Gene silencing by adenovirus-delivered siRNA. FEBS Lett. 539:111-4.
   Peptide Delivery:
Morris, M. C., L. Chaloin, F. Heitz, and G. Divita. 2000. Translocating peptides and proteins and their use for gene delivery. Curr Opin Biotechnol. 11:461-6.
Simeoni, F., M. C. Morris, F. Heitz, and G. Divita. 2003. Insight into the mechanism of the peptide-based gene delivery system MPG: implications for delivery of siRNA into mammalian cells. Nucleic Acids Res. 31:2717-24.

Other technologies that may be suitable for delivery of siRNA to the target cells are based on nanoparticles or nanocapsules such as those described in U.S. Pat. No. 6,649,192B and 5,843,509B.

Formulation and Administration

The nucleic acid of the invention can be formulated in pharmaceutical compositions. These compositions may comprise, in addition to the nucleic acid, a pharmaceutically acceptable excipient, carrier, buffer, stabiliser or other materials well known to those skilled in the art. Such materials should be non-toxic and should not interfere with the efficacy of the nucleic acid. The precise nature of the carrier or other material may depend on the route of administration, e.g. intravenous, cutaneous or subcutaneous, topical, intramuscular, or intraperitoneal routes.

The route of administration will depend on tumour type and location and the present invention is not limited to any particular route of administration. Administration may in particular be topical (for example for the treatment of melanoma and other tumours located on the skin), systemic (e.g. by parenteral administration), or by intratumoural injection.

For intravenous, cutaneous or subcutaneous injection, or intratumoural injection, the nucleic acid may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Administration is preferably in a “therapeutically effective amount”, this being sufficient to show benefit to the individual. The actual amount administered, and rate and time-course of administration, will depend on the nature and severity of the tumour being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

A composition may be administered alone or in combination with other treatments, either simultaneously or sequentially dependent upon the condition to be treated.

Suppression and Silencing

Nucleic acids of the invention are designed to suppress or silence the expression of Snail in a cell.

Suppression of expression results in a decrease in the quantity of Snail mRNA and protein. For example, in a given cell the suppression of Snail by administration of a nucleic acid of the invention results in a decrease in the quantity of Snail relative to an untreated cell.

Suppression may be partial. Preferred degrees of suppression are at least 50%, more preferably one of at least 60, 70, 80, 85 or 90%. A level of suppression between 90% and 100% is considered a ‘silencing’ of expression.

The invention includes the combination of the aspects and preferred features described except where such a combination is clearly impermissible or expressly avoided.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIG. 1A shows E-cadherin promoter activity (arbitrary units, 0-9) in MDCK-EGFP-Snail (1d) cells after transfection of pSuper-siEGFP construct (0, 250, 500, 750 and 1000 ng.

FIG. 1B shows E-cadherin promoter activity (arbitrary units, 0-1.2) in MDCK cells after co-transfection of pSuper-siEGFP construct (0, 0, 0, 750, 750 and 750 ng) and EGFP-Snail construct (0, 50, 100, 0, 50 and 100 ng).

FIG. 2A shows E-cadherin promoter activity (arbitrary units, 0-2.5) in MDCK-Snail cells after transfection of the indicated amounts of pSuper-siSnail 19 nt construct (0, 0, 250, 500, 750 and 1000 ng). The leftmost column represents the control, using wild-type MDCK cells.

FIG. 2B shows E-cadherin promoter activity (arbitrary units, 0-1.6) in wild-type MDCK cells after co-transfection of the indicated amounts of pSuper-siSnail 19 nt construct (0, 0, 0, 750, 750 and 750 ng) and EGFP-Snail construct (0, 50, 100, 0, 50 and 100 ng).

FIG. 2C shows the effect of pSuper-siEGFP (0, 0, 1000 and 0 ng) and pSuper-siSnail 19 nt (0, 0, 0 and 1000 ng) on EGFP-Slug mediated inhibition of E-cadherin promoter activity (arbitrary units, 0-1.4) in wild-type MDCK cells. EGFP-Slug: 0, 250, 250 and 250 ng

FIG. 3 shows E-cadherin promoter activity (arbitrary units, 0-1.6) in CarB cells after transfection of the indicated amounts (0, 0, 250, 500, 750 and 1000 ng) of pSuper-siSnail 19 nt construct. The leftmost column represents the control, using wild-type MDCK cells.

### DETAILED DESCRIPTION OF THE BEST MODE OF THE INVENTION

Specific details of the best mode contemplated by the inventors for carrying out the invention are set forth below, by way of example. It will be apparent to one skilled in the art that the present invention may be practised without limitation to these specific details.

The central hypothesis underlying the following experiments is that blockade of the interaction between Snail and its downstream targets, particularly E-Cadherin, will avoid the transition to the invasive phase of any given epithelial tumour, thus making Snail a suitable target for tumour therapy, particularly in early stage melanoma, breast or colon cancers.

Blockade of Snail expression is achieved by the use of specific siRNA oligonucleotides directed to specific Snail mRNA sequence, and without affecting expression of the highly related molecule, Slug.

### EXAMPLE 1

### siRNA Inhibits the Snail-Mediated Repression of E-Cadherin Promoter

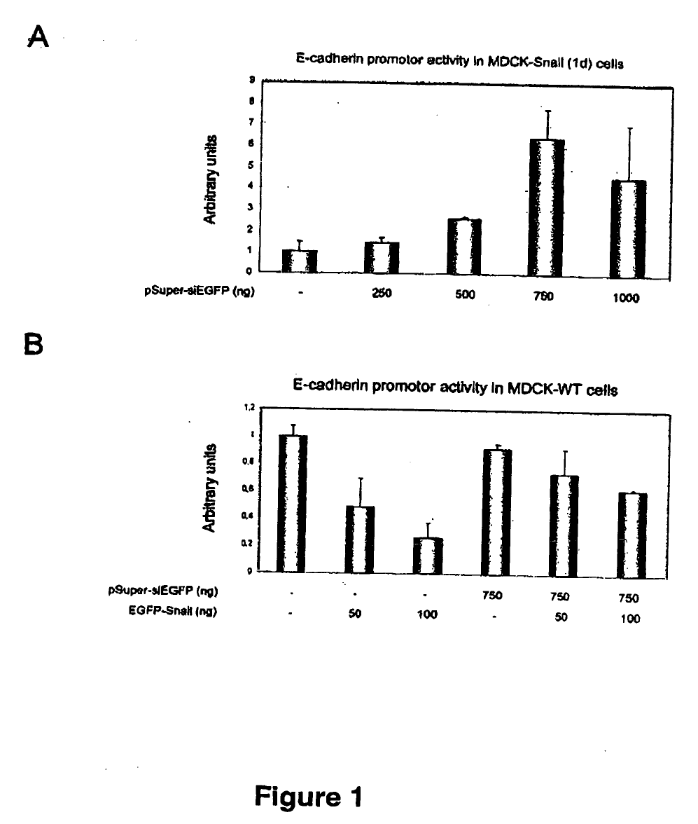

RNA interference technology was used to block Snail expression and the subsequent E-cadherin promoter repression. As a first stage, we developed EGFP-Snail fusion constructs and generated stable transfectant cell lines from MOCK cells. One of the MDCK-EGFP-Snail clones (1d) exhibited the EMT changes previously characterized in MDCK-Snail cell lines (Cano et al., 2000). 1d cells exhibit a fibroblastic-like phenotype, with complete absence of E-cadherin expression (data not shown) and maintain a stable low level of EGFP-Snail and repression of E-cadherin at the promoter level (FIG. 1A). Furthermore, association of EGFP-Snail to endogenous E-cadherin promoter in 1d cells has been conclusively detected by using chromatin immunuprecipitation analysis (Peinado et al., 2004).

Caplen et al, 2001 describes the design of siRNA oligonucleotides against a region of the GFP cDNA sequence. We cloned these siRNA oligonucleotides into the pSuper vector (Oligoengine). Transient transfection of siRNA-GFP-pSuper led to the derepression of E-cadherin promoter activity in 1d cells, inducing a 6 to 7-fold activation of the promoter (FIG. 1A), while it did not affect the promoter activity detected in control MDCK-WT cells (FIG. 1B).

In summary, Snail, expressed as a fusion protein with EGFP causes repression of the E-cadherin promoter. siRNA directed against the EGFP component of the fusion protein suppresses the expression of EGFP-Snail, leading to derepression of the E-cadherin promoter.

### EXAMPLE 2

### siRNA Against GFP Inhibits EMT Induced in MDCK Cells Expressing GFP-Snail

Stable transfectants of siRNA-GFP-pSuper in MDCK-EGFP-Snail (clone 1d) cells, co-transfected with a puromycin resistant vector to allow proper selection, were isolated and analysed by their phenotype and migratory behaviour. The generated siEGFP-cells (1d-siEGFP) showed a dramatic change in their phenotype, acquiring an epithelial morphology with apparent cell-cell contacts (data not shown). Immunofluorescence analysis of the 1d-siEGFP cells indicated re-expression of E-cadherin at cell-cell contacts and strong reduction in expression of vimentin, a typical marker of mesenchymal cells (data not shown). RT-PCR analyses indicated that stable transfection of pSuper-siEGFP construct induced the complete silencing of EGFP-Snail mRNA (data not shown). These results clearly indicate that suppression of EGFP-Snail expression induces the reversion of EMT, leading to reacquisition of an epithelial phenotype in MDCK-EGFP-Snail (1d) cells.

Because the EMT process induced by Snail is known to be associated with the acquisition of a motile phenotype (Cano et al., 2000), we next investigated the migratory behaviour of 1d-siEGFP cells and their parental 1d cells in a wound healing assay. 1d-si-EGFP cells close the wound by unidirectional coherent migration, in contrast to 1d cells that do it by random migration (data not shown), similarly to previously observed in MDCK-Snail cells (Cano et al., 2000). Furthermore, 1d-si-EGFP cells are unable to invade three-dimensional collagen gels, in contrast to 1d and MDCK-Snail cells (data not shown).

Taken together, these results indicate that silencing of Snail expression should be an appropriate strategy to revert suppression of E-cadherin expression and, therefore, for suppression of EMT and the invasive phenotype.

### EXAMPLE 3

### siRNA Specific to Snail Inhibits Snail-Mediated Repression of E-Cadherin Promoter in MDCK-Snail Cells

In order to prove that siRNA oligonucleotides specific to Snail mRNA are useful to repress endogenous Snail expression and its associated properties, we have designed a 19 nt siRNA to Snail mRNA. The sequence of the 19-mer nucleotide (GATGCACATCCGAAGCCAC, SEQ ID NO:1) is directed to the N-terminal region of the first zinc finger of Snail (position from 580 to 598 in NM_{—}005985, supra).

Importantly, this specific nucleotide sequence is conserved between mouse and human Snail mRNA sequence, but it is not conserved in Slug mRNA of any species (Sefton et al, 1998, Manzanares et al., 2001). Snail-Si-RNA-19 nt was cloned into the pSuper vector (Oligoengine) and its efficiency tested by analysis of its effect on E-cadherin promoter activity.

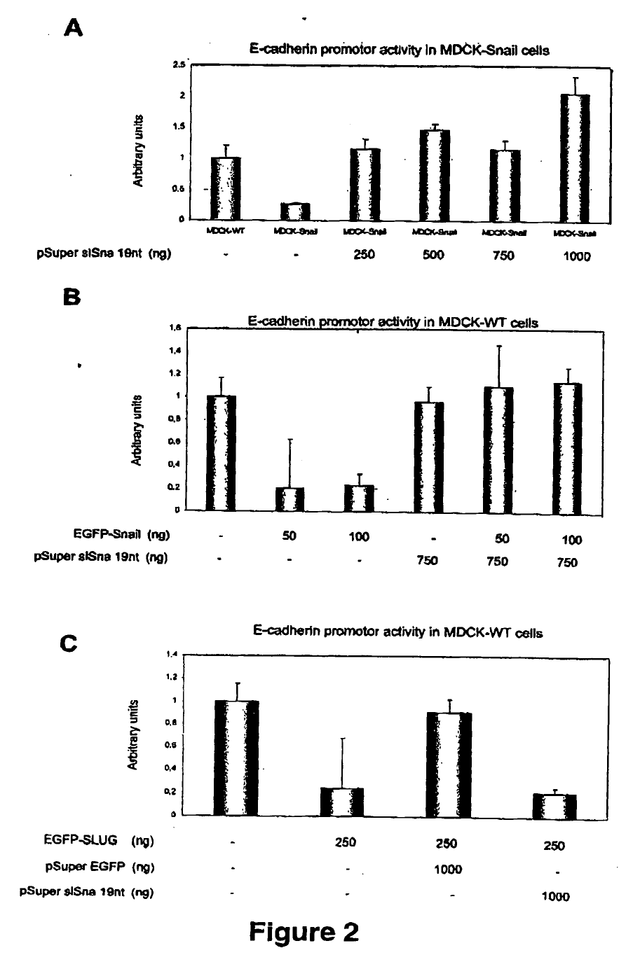

Co-transfection of Snail-Si-RNA-19 nt in MDCK-Snail cells, exhibiting very reduced endogenous E-cadherin activity (Cano et al, 2000), leads to strong induction of E-cadherin promoter, in a concentration dependent fashion, up to levels similar or even higher than that of control MDCK cells (FIG. 2A) To further confirm the efficiency of Snail-Si-RNA-19 nt, analysis of E-cadherin promoter were performed in control MDCK cells transiently co-transfected with Snail (in the EGFP-fusion form) in the absence or presence of Snail-Si-RNA-19 nt. As shown in FIG. 2B, co-transfection of EGFP-Snail induces a 75% repression of E-cadherin promoter after 24 h. However, this repression was fully abrogated by the presence of Snail-Si-RNA-19 nt. Importantly, Snail-si-RNA-19 nt does not have any effect by itself on the E-cadherin promoter activity in MDCK cells.

The specificity of Snail-Si-RNA-19 nt on Snail suppression was confirmed by analysis of its effect on E-cadherin repression mediated by the highly homologous factor Slug. The construction EGFP-Slug is able to induce a 75% repression of the E-cadherin promoter when co-transfected in MDCK cells (FIG. 2C), a result similar to that previously reported with Slug construct in the same cell system (Bolos et al., 2003). As expected, the EFGP-Slug mediated repression of E-cadherin promoter was fully relieved by co-transfection of the Si-RNA-EGFP indicating the efficient silencing of Si-EFGP-oligonucleotide. However, no effect was detected when Snail-si-RNA-19 nt was co-transfected with EGFP-Slug, since the activity of E-cadherin promoter remains with the same level of repression observed by co-transfection of EGFP-Slug alone, These results, strongly support the specificity of the designed Snail-si-RNA-19 nt to block Snail expression, without affecting other highly homologous factors, such as Slug.

Detailed Description of Snail-Si-RNA-19 nt

The complete oligonucleotide sequences used for cloning into pSuper were as follows.

Sense Oligonucleotide

(SEQ ID NO:9)

5′ -GATCCCCGATGCACATCCGAAGCCACTTCAAGAGAGTGGCTTCGGA
TGTGCATCTTTTTGGAAA-3′

The bold underlined sequence (GATGCACATCCGAAGCCAC, SEQ ID NO:1) corresponds to the 19mer target sequence of Snail mRNA.

The bold, non-underlined sequence (5′-GTGGCTTCGGATGTGCATC-3′, SEQ ID NO:10) is the reverse complement of the target sequence).

The non-bold, underlined 5′ sequence (GATC) is the BglII restriction sequence.

The non-bold, non-underlined central sequence (TTCAAGAGA, SEQ ID NO:11) is an unrelated spacer, which allows the formation of a hairpin in the transcribed mRNA).

The non-bold, underlined 3′ sequence (TTTTT) is an RNA pol III stop sequence.

The non-bold, non-underlined 5′ sequence (CCC) is the transcription initiation sequence.

Antisense Oligonucleotide

(SEQ ID NO:12)

5′ -AGCTTTTCCAAAAAGATGCACATCCGAAGCCACTCTCTTGAAGTGG
CTTCGGATGTGCATCGGG-3′

The bold sequences are complementary to the bold sequences of the sense oligonucleotide.

The non-bold, underlined 5′ sequence (AGCT) is the HindIII restriction sequence.

The non-bold, non-underlined central sequence (TCTCTTGAA, SEQ ID NO:13) is complementary to the hairpin sequence in the sense oligonucleotide.

The oligos were annealed in vitro and the cloned into the BglII/HindIII restriction sites of the pSuper-vector (OligoEngine).

The recombinant plasmid was transfected into cells using the lipofectamine method, as described in our previous papers (e.g. Cano et al., 2000; Bolós et al., 2003).

For stable transfections, we used co-transfection with a puromycin-resistance vector. (More recently, we are also using a similar strategy but cloning the oligos into the pSuperior-Puro vector (OligoEngine), which includes resistance to puromycin, allowing direct selection without co-transfection.)

Transcription from pSuper yields an RNA transcript having the sequence:

(SEQ ID NO:14)

5′ -GAUGCACAUCCGAAGCCACUUCAAGAGAGUGGCUUCGGAUGUGCAU
CUU-3′

The motifs are as described for the sense oligonucleotide.

The target sequence and complementary sequence then self-hybridise, to form an shRNA. (The 5′-U and 3′-A of the hairpin sequence may also hybridise.)

This is then processed by DICER into the dsRNA;

| | |
|---|---|
| 5′ -GAUGCACAUCCGAAGCCACUU-3′ | (SEQ ID NO:15) |
| 3′ -UUCUACGUGUAGGCUUCGGUG-5′ | |

The lower strand then targets Snail mRNA for degradation.

### EXAMPLE 3

### siRNA Specific to Snail Inhibits Snail-Mediated Repression of E-Cadherin Promoter in Carcinoma Cells

To obtain further evidence of the efficiency of Snail-Si-RNA-19 nt as a tool to specifically suppress endogenous Snail expression, we have analysed its effect on a previously characterized carcinoma cell line (CarB).

CarB cells are derived from a mouse skin spindle cell carcinoma, showing a fibroblastic morphology and highly tumorigenic and metastatic phenotype, and they are completely deficient in E-cadherin expression (Navarro et al., 1991). Furthermore, CarB cells show a complete repression of E-cadherin promoter activity (Faraldo et al., 1997) and express high levels of endogenous Snail and Slug factors (Cano et al., 2000). They also express E47 (Perez-Moreno et al., 2001).

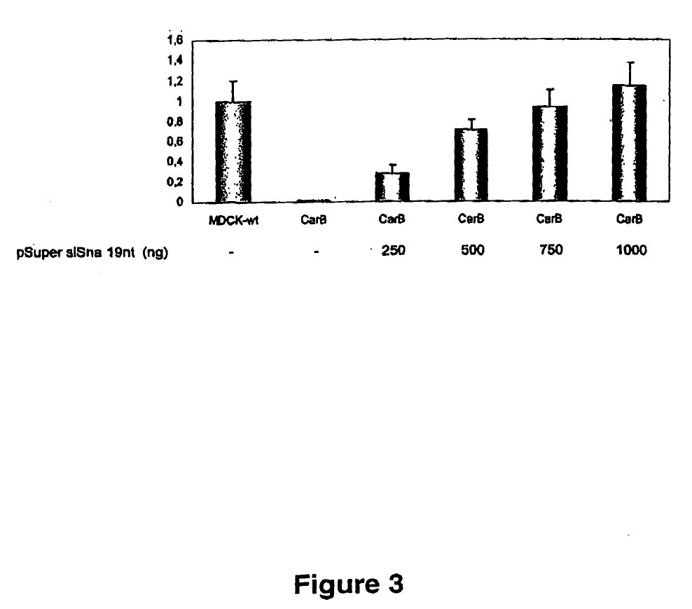

Analysis of E-cadherin promoter activity was performed in CarB cells in the absence or presence of Snail-Si-RNA-19 nt. As shown in FIG. 3, E-cadherin promoter activity of CarB cells is extremely low as compared to prototypic epithelial cells, such as MDCK. Co-transfection of Snail-Si-RNA-19 nt leads to a dramatic induction of E-cadherin promoter activity in CarB cells (FIG. 3), in a dose-dependent fashion. At the highest concentration used (750 and 1000 ng), Snail-Si-RNA-19 nt induces reactivation of E-cadherin promoter in CarB cells to levels similar to those obtained in epithelial MDCK cells (FIG. 3). In absolute terms, Snail-Si-RNA-19 nt induces a 200-fold activation of E-cadherin promoter in CarB cells.

These results provide compelling evidence that the use of siRNA oligonucleotides designed to the specific sequence of mouse/human Snail mRNA described here is a powerful tool to block endogenous Snail expression and its effects on E-cadherin repression. Therefore, they should be useful for blocking the subsequently associated processes of EMT and tumour invasion.

### EXAMPLE 4 (COMPARATIVE)

### Generation of a Different siRNA Against Snail

A similar strategy to that described above was used to generate a different siRNA directed against Snail mRNA.

In this case, we used a sequence derived from dog Snail mRNA (corresponding to positions 391-409 of the partial cds sequence: GenBank accession no. AF282628; version AF282628.1; GI:18000998), in a region fully identical to human Snail (positions 550-568 of NM_{—}005985, supra). It has a single nucleotide different from mouse Snail (positions 543-561 of NM_{—}011427, supra), and three different from both human Slug (positions 659-677 of NM_{—}003068, supra) and mouse Slug (positions 541-559 of NM_{—}011415, supra), which are identical in this region.

The target sequence was:
5′-CAAGGAATACCTCAGCCTG-3′ (SEQ ID NO:16)

Sense and antisense oligonucleotides were constructed and cloned into pSuper as described above.

In preliminary experiments, this siRNA construct did not suppress endogenous Snail mRNA in MDCK cells (Madin Darby Canine Kidney cells).

### EXAMPLE 5

### siRNA Specific to Snail Inhibits Tumour Growth In Vivo

Stable transfectants of CarB cells with Snail-si-RNA-19 nt in pSuperior were generated after selection with 1 μg/ml puromycin (siRNA derived cells) during two to four weeks. Ten to twenty clones were isolated and individually characterized for Snail, Slug and E-cadherin expression. Two of the clones (called C25 and C27) were chosen for in vivo analysis.

The in vivo effect of the inhibition of Snail expression in CarB cells was tested by orthotopic injection of CarB and CarB-siEGFP control cells or the stable clones CarB-siSnail-C25 and CarB-siSnail-C27 into nude mice. For that purpose, 10⁶ cells were intradermally injected into each flank of 6-8 week old nude mice. Two independent clones generated from CarB-siSnail cells (CarB-siSnail-C25 and CarB-siSnail-C27) were each tested on 8 animals and CarB cells were analysed in 4 animals. The tumor growth was estimated every 2-3 days by measuring the two orthogonal diameters with a calibre.

The mice injected with non-transformed CarB cells developed tumours significantly bigger than the mice transformed with CarB cells expressing Snail siRNA, demonstrating that Snail siRNA can block tumour progression.

After 14 days, average tumour size in mice injected with non-transformed CarB cells (>0.35 cm³) was more than twice that in mice infected with transformed cells (0.15 cm³).

Tumour non-progression was associated with re-expression of CDE, stabilisation and recruiting of beta-catenin to the cell membrane and a clear reduction in the expression of the extracellular protease MMP-9.

Although the foregoing invention has been described in some detail by way of illustration and example for purposes of clarity and understanding, it will be readily apparent to those of ordinary skill in the art in light of the teachings of this invention that certain changes and modifications may be made thereto without departing from the spirit or scope of the appended claims.

### REFERENCES

Batlle, E., et al. (2000) The transcription factor snail is a repressor of E-cadherin gene expression in epithelial tumour cells. Nat Cell Biol 2, 84-89
Behrens, J., et al. (1992) Cell adhesion in invasion and metastasis. Semin Cell Biol 3, 169-178
Birchmeier, W and Behrens, J. (1994) Cadherin expression in carcinomas: role in the formation of cell junctions and the prevention of invasiveness. Biochim Biophys Acta 1198, 11-26.
Blanco, M. J., et al. (2002), Correlation of Snail expression with histological grade and lymph node status in breast carcinomas, Oncogene 21, 3241-3246.
Bolos, V., et al. (2003) The transcription factor Slug represses E-cadherin expression and induces epithelial to mesenchymal transitions: a comparison with Snail and E47 repressors. J. Cell Sci., 116, 499-511.
Cano, A., et al. (2000). The transcription factor Snail controls epithelial-mesenchymal transitions by repressing E-cadherin expression. Nat Cell Biol 2, 76-83.
Carver, E. A., et al. (2001) The mouse snail gene encodes a key regulator of the epithelial-mesenchymal transition. Mol Cell Biol 21, 8184-8188.
Cheng, C. W., et al. (2001). Mechanisms of inactivation of E-cadherin in breast carcinoma: modification of the two-hit hypothesis of tumor suppressor gene. Oncogene 20, 3814-3823.
Christofori, G. and Semb, H. (1999). The role of the cell-adhesion molecule E-cadherin as a tumour-suppressor gene. Trends Biochem Sci 24, 73-76.
Comijn, J., et al. (2001). The two-handed E box binding zinc finger protein SIP1 downregulates E-cadherin and induces invasion. Mol Cell 7, 1267-1278
Czauderna, F., et al. (2003) Structural variations and stabilising modifications of synthetic siRNAs in mammalian cells. Nucleic Acids Res. 31, 2705-16.
Elbashir, S. M., et al. (2001a). Duplexes of 21-nucleotide RNAs mediate RNA interference in cultured mammalian cells, Nature 411, 494-8.
Elbashir, S. M., et al. (2001b). RNA interference is mediated by 21- and 22-nucleotide RNAs. Genes Dev. 15, 188-200
Elbashir, S. M., et al. (2001c). Functional anatomy of siRNAs for mediating efficient RNAi in Drosophila melanogaster embryo lysate. Embo J 20, 6877-88
Faraldo, M. L, et al. (1997). Analysis of the E-cadherin and P-cadherin promoters in murine keratinocyte cell lines from different stages of mouse skin carcinogenesis. Mol Carcinog 20, 33-47.
Giroldi, L, A., et al. (1997). Role of E boxes in the repression of E-cadherin expression. Biochem Biophys Res Commun 241,453-458.
Grooteclaes, M. L. and Frisch, S. M. (2000). Evidence for a function of CtBP in epithelial gene regulation and anoikis. Oncogene 19, 3823-3828.
Hajra, K. M., et al. (1999). Extinction of E-cadherin expression in breast cancer via a dominant repression pathway acting on proximal promoter elements. Oncogene 18, 7274-7279.
Hajra, K. M., et al. (2002). The SLUG zinc-finger protein represses E-cadherin in breast cancer. Cancer Res 62, 1613-1618.
Hennig, G., et al. (1996). Mechanisms identified in the transcriptional control of epithelial gene expression, J Biol Chem 271, 595-602.
Huber, O., et al. (1996). Cadherins and catenins in development. Curr Opin Cell Biol 8, 685-691,
Nieto, M. A. (2002). The Snail superfamily of zinc-finger transcription factors. Nat Rev Mol Cell Biol 3, 155-166.
Perez-Moreno, et al. (2001). A new role for E12/E47 in the repression of E-cadherin expression and epithelial-mesenchymal transitions. J Biol Chem 276, 27424-27431.
Perl, A. K., et al. (1998). A causal role for E-cadherin in the transition from adenoma to carcinoma. Nature 392, 190-193.
Poser, I., et al. (2001). Loss of E-cadherin expression in melanoma cells involves up-regulation of the transcriptional repressor Snail. J Biol Chem 276, 24661-24666.

Risinger, J. I., et al. (1994). Mutations of the E-cadherin gene in human gynecologic cancers. Nat Genet 7, 98-102.
Rodrigo, I., et al. (1999). Regulation of E-cadherin gene expression during tumor progression: the role of a new Ets-binding site and the E-pal element, Exp Cell Res 248, 358-371.
Stetler-Stevenson, W. G., et al. (1993). Tumor cell interactions with the extracellular matrix during invasion and metastasis. Annu Rev Cell Biol 9, 541-573.
Sugimachi, K., et al. 2003. Transcriptional repressor snail and progression of human hepatocellular carcinoma. Clin Cancer Res, 9:2657-2664.
Takechi, M. (1993) Cadherins in cancer: implications for invasion and metastasis. Curr Opin Cell Biol 5, 806-811.
Takechi, M. (1995) Morphogenetic roles of classic cadherins. Curr Opin Cell Biol 7, 619-627.
Thiery, J P (2002) Epithelial-mesenchymal transitions in tumour progression. Nat. Rev. Cancer 2:442-454
Yokohama, K. et al. (2001) reverse correlation of E-cadherin and Snail expression in oral squamous cell carcinoma cells in vitro. Oral Oncol. 37, 65-71.
Yoshiura, K., et al. (1995). Silencing of the E-cadherin invasion-suppressor gene by CpG methylation in human carcinomas. Proc Natl Acad Sci USA 92, 7416-7419.
Caplen, N. J., et al. (2001). Specific inhibition of gene expression by small double-stranded RNAs in invertebrate and vertebrate systems Proc Natl Acad Sci USA 98, 9742-9747.
Manzanares, M. A., et al. 2001. The increasing complexity of the Snail gene superfamily in metazoan evolution. Trends Genet. 17:178-181.
Navarro, P. M. et al. (1991). A role for the E-cadherin cell-cell adhesion molecule in tumor progression of mouse epidermal carcinogenesis. J. Cell Biol. 115:517-533.
Peinado, H., et al. (2004). Snail mediates E-cadherin repression by the recruitment of the Sin3A/Histone Deacetylase 1/2 complex. Mol. Cell. Biol. 24:306-319.
Sefton, M., et al. 1998. Conserved and divergent roles for members of the Snail family of transcription factors in the chick and mouse embryo. Development. 125:3111-3121.

These and all other publications referred to in the specification are incorporated herein by reference in their entirety and for all purposes.

1. Nucleic acid that is capable, when suitably introduced into or expressed within a mammalian cell that otherwise expresses Snail, of suppressing Snail expression by RNAi, wherein the nucleic acid targets a sequence of human Snail mRNA, as defined in GenBank accession no. NM_{—}005985 (version NM_{—}005985.2; GI:18765740; SEQ ID NO:28), that overlaps with the sequence gatgcacatccgaagccac (SEQ ID NO:1).

2. Nucleic acid according to claim 1 that targets a sequence of human Snail mRNA (SEQ ID NO:28) that is slightly longer or shorter than, but otherwise identical to, SEQ ID NO:1.

3. Nucleic acid according to claim 2, wherein the target sequence is 17-23 nucleotides in length.

4. Nucleic acid according to claim 1, wherein the target sequence overlaps with SEQ ID NO:1 by at least 5 nucleotides at either end of SEQ ID NO:1.

5. Nucleic acid according to claim 4, wherein the target sequence overlaps with SEQ ID NO: 1 by at least 10 nucleotides at either end of SEQ ID NO:1.

6. Nucleic acid according to claim 5, wherein the target sequence overlaps with SEQ ID NO; 1 by at least 15 nucleotides at either end of SEQ ID NO:1.

7. Nucleic acid according to claim 1, comprising a double-stranded siRNA of the following structure:

| | |
|---|---|
| 5′- X-Y -3′ | (SEQ ID NO:7) |
| 3′- Y′-X′ -5′ | |

wherein:
X is the RNA sequence 5′-W-UGCACAUCCGAAGCC-Z-3′ (SEQ ID NO:8), or an RNA sequence differing therefrom by a single substitution;
W is absent or is an RNA sequence selected from the group consisting of A, GA, AGA, AAGA, CAAGA, or UCAAGA;
Z is absent or is an RNA sequence selected from the group consisting of A, AC, ACA, ACAC, ACACG, or ACACGC;
W and Z together have between 2 and 8 nucleotides;
X′ is the reverse complement of X; and
Y and Y′ are absent or are each an RNA or DNA dinucleotide and may be the same or different.

8. Nucleic acid according to claim 7, wherein W is GA; Z is AC; Y and Y′ are UU or TT.

9. Nucleic acid according to claim 1, comprising a double-stranded siRNA that includes a double-stranded RNA sequence selected from the following group:

| | |
|---|---|
| 5′ -GAUGCACAUCCGAAGCC-3′ | (SEQ ID NO:3) |
| 3′ -CUACGUGUAGGCUUCGG-5′ | |
| 5′ -GAUGCACAUCCGAAGCCA-3′ | (SEQ ID NO:4) |
| 3′ -CUACGUGUAGGCUUCGGU-5′ | |
| 5′ -GAUGCACAUCCGAAGCCAC-3′ | (SEQ ID NO:2) |
| 3′ -CUACGUGUAGGCUUCGGUG-5′ | |
| 5′ -GAUGCACAUCCGAAGCCACA-3′ | (SEQ ID NO:5) |
| 3′ -CUACGUGUAGGCUUCGGUGU-5′ | |
| 5′ -GAUGCACAUCCGAAGCCACAC-3′ | (SEQ ID NO:6) |
| 3′ -CUACGUGUAGGCUUCGGUGUG-5′ | |
| 5′ -GAUGCACAUCCGAAGCCACACG-3′ | (SEQ ID NO:18) |
| 3′ -CUACGUGUAGGCUUCGGUGUGC-5′ | |
| 5′ -GAUGCACAUCCGAAGCCACACGC-3′ | (SEQ ID NO:19) |
| 3′ -CUACGUGUAGGCUUCGGUGUGCG-5′ | |
| 5′ -UGCACAUCCGAAGCCAC-3′ | (SEQ ID NO:22) |
| 3′ -ACGUGUAGGCUUCGGUG-5′ | |
| 5′ -AUGCACAUCCGAAGCCAC-3′ | (SEQ ID NO:23) |
| 3′ -UACGUGUAGGCUUCGGUG-5′ | |
| 5′ -AGAUGCACAUCCGAAGCCAC-3′ | (SEQ ID NO:24) |
| 3′ -UCUACGUGUAGGCUUCGGUG-5′ | |
| 5′ -AAGAUGCACAUCCGAAGCCAC-3′ | (SEQ ID NO:25) |
| 3′ -UUCUACGUGUAGGCUUCGGUG-5′ | |
| 5′ -CAAGAUGCACAUCCGAAGCCAC-3 | (SEQ ID NO:26) |
| 3′ -GUUCUACGUGUAGGCUUCGGUG-5′ | |
| 5′ -UCAAGAUGCACAUCCGAAGCCAC-3′ | (SEQ ID NO:27) |
| 3′ -AGUUCUACGUGUAGGCUUCGGUG-5′ | |

and a double stranded RNA sequence having a single base pair substitution compared to any one of SEQ ID NOs 2, 3-6, 18, 19 and 22-27.

10. A nucleic acid according to claim 1, comprising a double-stranded siRNA that includes the double-stranded RNA sequence

| | |
|---|---|
| 5′ -GAUGCACAUCCGAAGCCAC-3′ | (SEQ ID NO:2) |
| 3′ -CUACGUGUAGGCUUCGGUG-5′. | |

11. A double-stranded siRNA according to claim 10, which has 3′ dinucleotide DNA or RNA overhangs.

12. Nucleic acid according to claim 1 comprising DNA that, when transcribed in a mammalian cell, yields an shRNA having two complementary portions which are capable of self-hybridising to produce a double-stranded motif including a sequence selected from the group consisting of SEQ ID NOs: 2-6, 18, 19 and 22-27 or a sequence that differs from any one of the aforementioned sequences by a single base pair substitution.

13. Nucleic acid according to claim 12, wherein the complementary portions are joined by a spacer.

14. Nucleic acid according to claim 13, wherein the spacer is 4-12 nucleotides in length.

15. Nucleic acid according to claim 14, wherein the spacer is 6-9 nucleotides in length.

16. Nucleic acid according to claim 13, wherein the 5′ end of the spacer consists of the nucleotides -UU- or -UG-.

17. Nucleic acid according to claim 13, wherein the spacer consists of the nucleotides UUCAAGAGA.

18. Nucleic acid according to claim 12, wherein the transcribed RNA includes a 3′ overhang from the downstream complementary portion.

19. Nucleic acid according to claim 18, wherein the overhang is -UU or -UG.

20. Nucleic acid according to claim 12, wherein the shRNA has the sequence:
(SEQ ID NO:14)
5′ -GAUGCACAUCCGAAGCCACUUCAAGAGAGUGGCUUCGGAUGUGCAU
CUU-3′,
wherein,
the sequence GAUGCACAUCCGAAGCCAC (SEQ ID NO:1) corresponds to the 19mer target sequence of Snail mRNA;
the sequence 5′-GUGGCUUCGGAUGUGCAUC-3′ (SEQ ID NO:10) is the reverse complement of the target sequence;
the sequence UUCAAGAGA (EQ ID NO:11) is an unrelated spacer, which allows the formation of a hairpin in the transcribed mRNA.

21. A nucleic acid vector comprising the nucleic acid of claim 12, together with control sequences for directing its transcription in a mammalian cell.

22. A method of suppressing Snail expression in a mammalian tumour cell, the method comprising administering to the cell a nucleic acid according to claim 1.

23. A method of suppressing Snail expression in a mammalian tumour cell, the method comprising administering to the cell a nucleic acid according to claim 7.

24. A method of suppressing Snail expression in a mammalian tumour cell, the method comprising administering to the cell a nucleic acid according to claim 9.

25. A method of suppressing Snail expression in a mammalian tumour cell, the method comprising administering to the cell a nucleic acid according to claim 12.

26. A method of suppressing Snail expression in a mammalian tumour cell, the method comprising administering to the cell a nucleic acid according to claim 20.

27. A method of suppressing Snail expression in a mammalian tumour cell, the method comprising administering to the cell a vector according to claim 21.

28. A method according to claim 22, wherein the tumour is a carcinoma or melanoma.

29. A method of derepressing the E-cadherin promoter in a mammalian tumour cell, the method comprising administering to the cell a nucleic acid according to claim 1.

30. A method of derepressing the E-cadherin promoter in a mammalian tumour cell, the method comprising administering to the cell a nucleic acid according to claim 7.

31. A method of derepressing the E-cadherin promoter in a mammalian tumour cell, the method comprising administering to the cell a nucleic acid according to claim 9.

32. A method of derepressing the E-cadherin promoter in a mammalian tumour cell, the method comprising administering to the cell a nucleic acid according to claim 12.

33. A method of derepressing the E-cadherin promoter in a mammalian tumour cell, the method comprising administering to the cell a nucleic acid according to claim 20.

34. A method of derepressing the E-cadherin promoter in a mammalian tumour cell, the method comprising administering to the cell a vector according to claim 21.

35. A method according to claim 29, wherein the tumour is a carcinoma or melanoma.

36. A composition comprising a nucleic acid according to claim 1 in admixture with one or more pharmaceutically acceptable carriers.

37. A composition comprising a nucleic acid according to claim 7 in admixture with one or more pharmaceutically acceptable carriers.

38. A composition comprising a nucleic acid according to claim 9 in admixture with one or more pharmaceutically acceptable carriers.

39. A composition comprising a nucleic acid according to claim 12 in admixture with one or more pharmaceutically acceptable carriers.

40. A composition comprising a nucleic acid according to claim 20 in admixture with one or more pharmaceutically acceptable carriers.

41. A composition comprising a vector according to claim 21 in admixture with one or more pharmaceutically acceptable carriers.

42. A composition according to claim 41, wherein the carrier(s) include a lipophilic carrier or vesicle.
